(19) United States
(12) Patent Application Publication
(10) Pub. No.: US 2026/0054006 A1
(43) Pub. Date: Feb. 26, 2026

(54) DISPOSABLE PISTON-DRIVEN CASSETTE FOR INFUSION SYSTEMS

(71) Applicant: CareFusion 303, Inc., San Diego, CA (US)

(72) Inventor: Daniel PETT, Vista, CA (US)

(21) Appl. No.: 18/812,960

(22) Filed: Aug. 22, 2024

(51) Int. Cl. A61M 5/142; A61M 5/168

(52) U.S. Cl. CPC A61M 5/1422; A61M 5/16804; A61M 2005/14208; A61M 2205/12; A61M 2205/3334

(57) ABSTRACT

A pump cassette is configured to be seated in, and connect to, a cassette recess of an infusion pump system. A cassette drive interface of the cassette is configured to interface with a corresponding pump drive interface provided by the infusion pump system when the cassette is seated in and connected to the cassette recess. The cassette includes multiple pistons that are operatively connected to the cassette drive interface and configured to, when the housing is seated in the cassette recess of the infusion pump system and the pump drive interface is actuated by the infusion pump system, operate in concert with each other to continuously move a fluid received from the inlet port to the outlet port.

### TECHNICAL FIELD

The present disclosure generally relates to apparatus, systems, and methods of delivering medical fluid to patients, and more particularly to disposable cassettes for infusion pump systems.

### BACKGROUND

Infusion pumps are medical devices that may be used to administer intravenous (IV) fluids. An infusion pump can facilitate the delivery of IV fluids while controlling the volumes and rates for the delivery of such IV fluids. The IV fluids may be delivered at continuous rates or intermittent intervals. While piston pumps, particularly dual cylinder pumps, have been found to provide accurate flow rates under continuous pumping circumstances, such pumps have not been used in a medical setting because the pistons are in direct contact with the fluid, preventing reusability between patients. Instead, infusion pumps move fluid through an IV tube using a peristaltic pumping mechanism that externally acts on the IV tube to move the IV fluid from a container to a patient. The IV tube is typically connected to or integrated with an IV set (e.g., tubing, valves, and fittings for delivering fluid to a patient), and therefore the IV set may be disposable to reduce the risk of infection and contamination.

### SUMMARY

The subject technology provides a disposable IV pump cassette that incorporates a dual piston configuration for use in an infusion pump system. In accordance with certain aspects, a pump cassette comprises a cassette housing comprising an inlet port to an outlet port, the cassette housing being configured to be seated in, and connect to, a cassette recess of an infusion pump system; a cassette drive interface configured to interface with a corresponding pump drive interface provided by the infusion pump system when the cassette housing is seated in and connected to the cassette recess; and multiple pistons and corresponding piston chambers disposed within the cassette housing and fluidly connected between the inlet port and the outlet port, each of the multiple pistons being operatively connected to the cassette drive interface and configured to, when the housing is seated in the cassette recess of the infusion pump and the pump drive interface is actuated by the infusion pump, operate in concert with each other to continuously move a fluid received from the inlet port to the outlet port, such that a first piston of the multiple pistons delivers a first portion of the fluid received into a first piston chamber of the corresponding piston chambers to the outlet port while a second piston of the multiple pistons draws a second portion of the fluid into a second piston chamber of the corresponding piston chambers from the inlet port, and the second piston delivers the second portion of the fluid from the second piston chamber to the outlet port while the first piston draws a third portion of the fluid into the first piston chamber from the inlet port, wherein a controllable fluid pathway is formed between the inlet port and the outlet port and includes the first and second piston chambers.

A piston-driven cassette based infusion system comprises the pump cassette and the infusion pump system comprising a pump housing, wherein the pump housing comprises the cassette recess, and wherein the infusion pump is configured to actuate the pump drive interface to cause the fluid to move through the controllable fluid pathway.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts a first example infusion pump system for use with a dual piston pump cassette, according to various aspects of the subject technology. FIG. 1B depicts a second example infusion pump system for use with a dual piston pump cassette, according to aspects of the subject technology.

FIG. 2 depicts a pump cassette and a corresponding cassette recess of an infusion pump system, according to aspects of the subject technology.

FIG. 3 depicts an example disposable IV pump cassette, according to aspects of the subject technology.

FIG. 4 depicts an example process for operating a piston-driven cassette based infusion system, according to various aspects of the subject technology.

FIG. 5 is a conceptual diagram illustrating an example electronic system for operating a piston-driven cassette based infusion system, according to aspects of the subject technology.

### DETAILED DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The subject technology involves a pump cassette technology for use in large volume infusions. The pumping mechanism of the infusion pump is built into a disposable part, a housing that includes an inlet, an outlet, and internal pistons for pumping a fluid through the disposable part. According to various implementations, parallel pistons and cylinders operate, within the disposable unit, in an alternating fashion, aspirating and delivering medication. This system allows for more precise flow rate accuracy due to known volume of each cylinder and volume or medication delivered for each stroke/rotation of the motor in the pump, while providing continuous flow that does not pulse. The fluid path remains closed and intact, and the entire path of the fluid, including the pumping mechanism remains fluidly isolated from the electrical systems of the overall infusion pump system. Air bubbles are forced out of the cylinder with each compression, thus also reducing air-in-line issues. In this regard, the subject technology simplifies conventional infusion pump systems by requiring only a driving interface for the cassette, such as a rotation motor that can vary in speed.

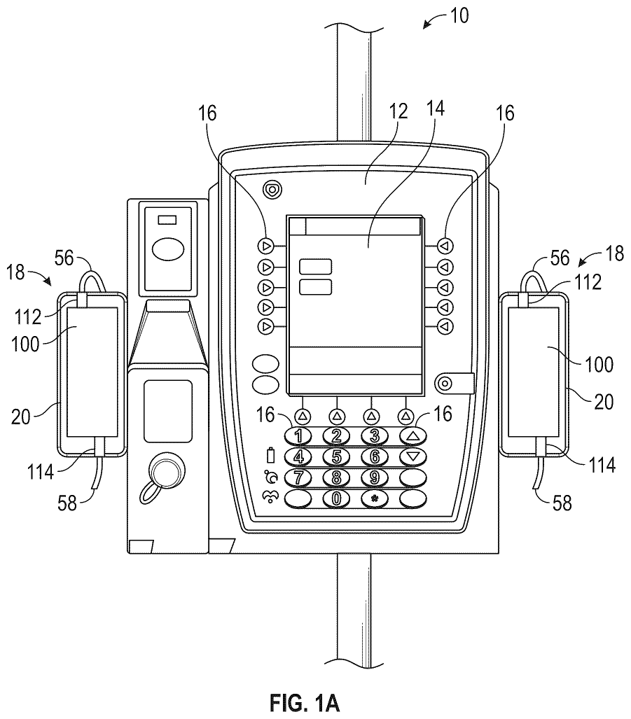

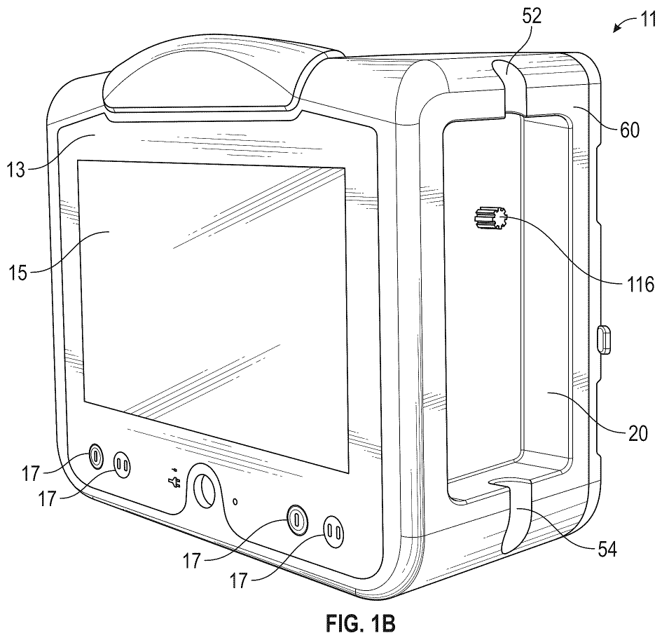

FIG. 1A depicts a first example infusion pump system 10 for use with a dual piston pump cassette, according to various aspects of the subject technology. It is to be understood that this is only an exemplary infusion pump system, and the dual piston pump cassette (or cartridge) 100 can be utilized in any type of infusion pump system. For example, while cassette recesses 20 are depicted in FIG. 1A as being implemented as part of interface modules 18, the cassette recesses 20 may be implemented as part of processing unit 12 or another device associated with the infusion pump system 10, and are described herein irrespective of a host device.

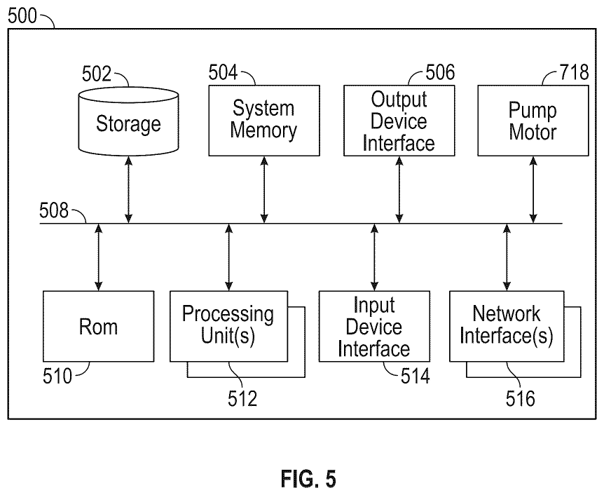

The infusion pump system will be generally explained in reference to FIGS. 1-5. An exemplary infusion pump system 10 may include central processing unit 12 with display screen 14 (e.g., touchscreen display), and data input features 16, for example, a keypad and a series of configurable buttons adjacent to display screen 14. The processing unit 12 may incorporate one or more processors, storage devices, input/output devices, network devices, and the like, as described with respect to FIG. 5. As will be described further, the processing unit 12 or the interface module 18 may include a motor (see FIG. 5) for driving multiple pistons incorporated into a disposable cassette 100. An upstream infusion line 56 is connected to an inlet port 112 of the cassette 100 and a downstream infusion line 58 (e.g., which may also be connected to a patient) is connected to an outlet port 114 of the cassette 100.

FIG. 1B depicts a second example infusion pump system for use with a dual piston pump cassette, according to aspects of the subject technology. Infusion pump system 11 may include central processing unit 13 with display screen 15 (e.g., touchscreen display), and data input features 17, for example, a series of configurable buttons adjacent to display screen 15. In some implementations, the display screen 15 may provide a keypad or similar data entry feature. Other types of input and output devices may be used with central processing unit 13 and infusion pump system 11. In certain aspects, central processing unit 13 is operatively coupled to one or more interface modules, with cassette recesses 20, to control and communicate with various operational interfaces thereof.

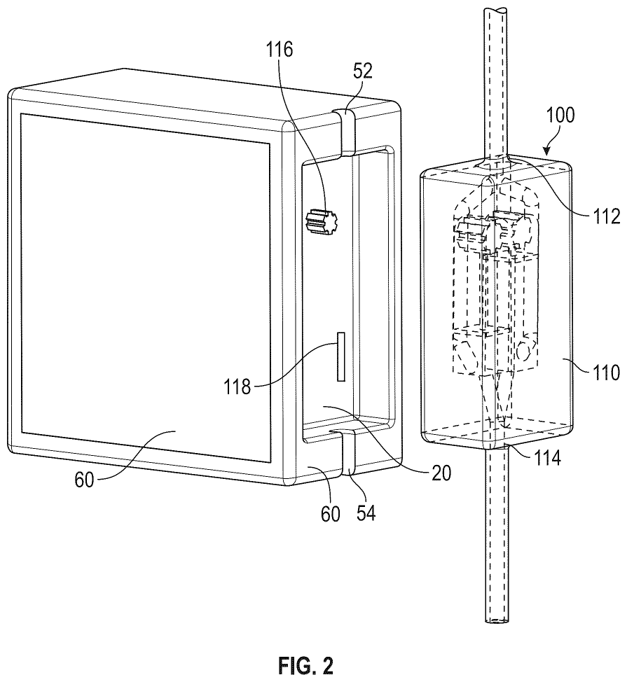

In the depicted example, the exemplary infusion pump system 11 includes a cassette recess 20 formed within a housing 60 of the infusion pump system 10 (e.g., within the housing of processing unit 12). The recess 20 is configured to receive a disposable IV pump cassette, such as the pump cassette 100 depicted in FIG. 2. The cassette recess 20 may be configured to provide various mechanical couplings and operational interfaces (e.g., fittings, motor, gearing, driveshaft, sensors, etc.). As will be described further, a pump driving interface 116 is positioned within the recess 20 to, when a pump cassette is seated within the recess, transfer a rotational energy to a cassette drive interface of the pump cassette causing, based on the rotational energy, multiple pistons and corresponding piston chambers disposed within (e.g., sealed within) the cassette housing to operate in concert with each other to continuously move a fluid received from an inlet port to an outlet port.

In the depicted example, the housing 60 includes further respective indentations and/or recesses 52, 54 for receiving at least a portion of a source infusion line 56 and a delivery infusion line 58, respectively. In this regard, when the cassette is seated into the cassette recess 20, the corresponding source and delivery lines, which may stem from the top and bottom of the pump cassette, will not prevent the cassette from being seated and will be relatively flush with the outer housing of infusion system. The indentations/recesses 52, 54 further provide for stability of the entire pump cassette by anchoring the pump cassette by way of capturing the source and delivery lines.

FIG. 2 depicts a pump cassette 100 and a corresponding cassette recess 20 of an infusion pump system, according to aspects of the subject technology. As shown, the cassette recess 20 is formed within a housing of the infusion pump system.

The pump cassette 100 includes a cassette housing 110 and the housing includes an inlet port 112 and an outlet port 114 (which may or may not extend from the housing 110). The cassette housing is specifically configured to be seated in, and connect to, the cassette recess 20. While the cassette housing 110 is described herein as having certain configurations of components, including the pistons within the housing, it is understood that the housing, and the components therein, may vary in size, depending on care requirements. For example, the chambers of the disclosed pistons may vary in size to accommodate different flow rates and/or volumes required for different care areas. That is, a neonatal cassette may be smaller than a cassette for adult patients. According to some implementations, a width and height of each different housing may remain constant to accommodate interchangeability between pump systems 10, but the depth of the housing may vary depending on the size of the components therein (e.g., based on volume and/or flow rate requirements).

According to various implementations, the cassette recess 20 includes a pump drive interface 116 configured to mechanically couple to a corresponding cassette drive interface 120 (shown in FIG. 2) of the pump cassette 100 to drive the pistons within the cassette 100. In some implementations, both interfaces are geared couplers configured to couple together by way of interlocking gears. For example, the pump drive interface 116 may include a drive shaft connected to a motor and having external gears circumscribing the shaft, and the cassette drive interface 120 may receive the drive shaft and have internal gears that couple to the external gears of the drive shaft (or vice versa). In this regard, a rotation of the pump drive interface 116 causes a rotation of the cassette drive interface 120, which causes, by way of a series of gears internal to the cassette 100, a configuration of internal pistons within the cassette 100 to pump fluid from the inlet 112 to the outlet 114.

In some implementations, the housing or cassette body 110 of the cassette 100 may include a handle portion and a plurality of protrusions or lugs that are configured to be releasably lockable with a plurality of slots of the cassette recess 20 (e.g., L-shaped locking channels). In this regard, cassette 100 can be self-latched into the cassette recess 20. Accordingly, a door or lever action is not required in order to retain the cassette 100 within the cassette recess 200. In an alternative embodiment, an inverse configuration may be desired, in which the cassette recess 200 would contain protrusions or lugs that would be configured to be releasably lockable with a corresponding slots located on the slider or rigid body.

The cassette body 110, or a substantial portion thereof, may extend a depth (D) between 6 mm and 12 mm into the recess 100. Fluid pathway extension member 128 may also extend between 6 mm to 12 mm. It is to be appreciated that the process of cleaning of upstream inlet recess 52, downstream outlet recess 54, and cassette recess 20 is made efficient in the shallow recess configuration in accordance with certain embodiments should any fluid or debris accumulate within cassette recess 20. The shallow recess configuration of cassette recess 20, and associated longitudinal alignment of cassette 100 such that a smaller of volumetric dimensions of cassette 100 (e.g., depth being smaller than length and/or width) further enables additional space for arrangement of mechanical couplings and operational interfaces and optimizes the overall space requirements of cassette recess 20 and infusion pump system 10 in general.

In operation, cassette 100 can be loaded (e.g., seated) directly into cassette recess 20. In this regard, the direct loading of the cassette 100 will enable avoidance of sheer forces that might otherwise be applied to the sensors, alignment features, and other engaging interfaces of cassette-facing surface of cassette recess 20 from interaction with the interface-facing side of cassette body 110 as it is loaded into cassette recess 20.

In some implementations, the cassette recess 20 includes one or more identification receiving sensors 118 that are, when the pump cassette is seated in the cassette recess, configured to interface with one or more corresponding cassette identification features 134 (shown in FIG. 2) coupled to or integrated with the cassette housing (e.g., on the back side of the housing, facing the recess 20). When the pump cassette 100 is seated in the cassette recess 20, the one or more identification receiving sensors 118 cause the infusion pump system 10 to identify, based on the one or more cassette identification features 134, one or more characteristics of the pump cassette 100. These characteristics may include, for example, a gear ratio of the gears within the cassette 100, a stroke volume of the piston(s) within the cassette 100, a range of flow rates that the cassette 100 is capable of providing, and the like.

For the purpose of this disclosure, actions (e.g., identifications, determinations, detections) described as being performed by the infusion pump system 10 may be determined by circuitry within the infusion pump system 10, including one or more processors and/or other circuitry and systems.

Additionally or in the alternative, the pump drive interface 100 may provide a variable power to the cassette drive interface. In some implementations, the motor causing the motion of the pistons within the cassette 100 may be integrated within the cassette 100, and the cassette drive interface may be configured to receive the variable power from the pump drive interface to power the motor within the cassette 100. In such instances, the processor of the infusion pump system 100 may control the flow rate within the pump by way of varying the power provided to the cassette drive interface.

In operation, an IV bag, syringe or other fluid source may be fluidly connected to inlet 112 of cassette 100, and outlet 114 of cassette 100 may be fluidly connected to a patient. In operation, a user (e.g., a caregiver) may obtain a new disposable IV cassette 100 and prime cassette 100. Due to the internal piston design, air bubbles in the line will enter the piston chamber and raise to the top and be captured in the top portion of the plunger chamber, thereby minimizing air-in-line errors.

According to various implementations, the infusion pump system 10 may include one or more pressure sensors to measure pressure within the infusion line and/or fluid pathway between inlet port 112 and outlet port 114. In some implementations, an upstream pressure sensor is positioned within the upstream inlet recess 52 to measure pressure within the source infusion line connected to the inlet port 112 of the cassette 100. In some implementations, an upstream pressure sensor is positioned within the downstream outlet recess 54 to measure pressure within the delivery infusion line connected to the outlet port 114 of the cassette 100.

In some implementations, the infusion pump system 10 can measure, when a pressure sensor is activated, a pressure in an infusion line fluidly coupled to the inlet or outlet port, and determine the error condition in part based on a pressure measured by the pressure sensor. Accordingly, in further implementations, the infusion pump system 10 can determine that the pressure satisfies a threshold pressure and, responsive the pressure satisfying the threshold pressure, suspend actuation of the pump drive interface to prevent further pumping of the fluid through the controllable fluid pathway.

Additionally or in the alternative, the infusion pump system 10 may be configured to monitor a resistance (or torque) encountered by the pump drive interface 116. For example, the infusion pump system 10 may detect a resistance (or torque) of the cassette drive interface 120 upon a rotation of the pump drive interface 116. When a threshold resistance (or torque) is reached, the infusion pump system 10 may determine that the error condition has occurred. In this regard, the error condition may include, for example, an occlusion occurring in an infusion line that is upstream or downstream of the cassette, or within thin cassette itself. In some implementations, responsive to detecting the threshold resistance (or torque), the infusion pump system may activate the pressure sensor(s), for example to confirm an increase (or drop) in pressure, and/or to diagnose the location of the occlusion. For example, a drop in pressure measured by the downstream pressure sensor may be indicative of an occlusion within the cassette or upstream of the cassette, while an increase in pressure measured by the downstream pressure sensor may be indicative of an occlusion downstream of the cassette. Accordingly, the error condition may be determined in part based on the pressure measured by one or more of the pressure sensors.

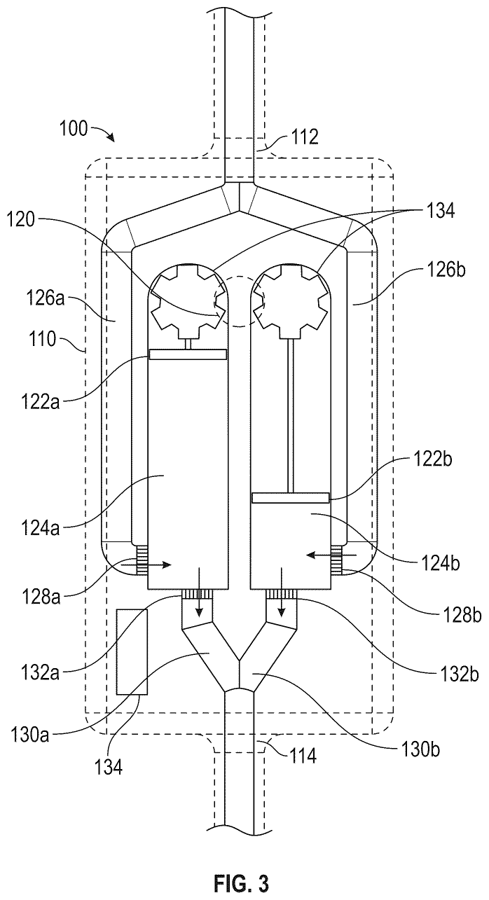

FIG. 3 depicts an example disposable IV pump cassette 100, according to aspects of the subject technology. The pump cassette 100 is shown from the rear, or recess 20 facing side of the cassette. As shown, the pump cassette 100 includes a cassette housing with an inlet port and an outlet port. A cassette drive interface 120 is positioned on the recess-facing side, and being configured to interface with a corresponding pump drive interface 116 provided by the infusion pump system 10 when the cassette housing is seated in and connected to the cassette recess.

Within the housing, or cassette body 110, the cassette 100 includes multiple pistons 122 a-b and corresponding piston chambers 124 a-b fluidly connected between the inlet port 112 and the outlet port 114. According to various implementations, each of the pistons are operatively connected to the cassette drive interface 120 and configured to, when the housing is seated in the cassette recess 20 of the infusion pump system 10 and the pump drive interface 116 is actuated (e.g., rotated or powered) by the infusion pump system 10, operate in concert with each other to continuously move a fluid received from the inlet port 112 to the outlet port 114.

Accordingly, the pistons may operate in parallel but pump inversely to each other to cause a continuous flow. That is, a first piston 112 a delivers a first portion of the fluid received into the first piston chamber 124 a to the outlet port 114 while the second piston 122 b draws a second portion of the fluid into the second piston chamber 124 b from the inlet port 112, and the second piston 122 a delivers the second portion of the fluid from the second piston chamber 124 a to the outlet port 114 while the first piston 122 a draws a third portion of the fluid into the first piston chamber 124 a from the inlet port 112.

Internally, in the depicted example, a first fluid pathway 126 a is fluidly coupled between the inlet port 112 and a fluid input 128 a of the first piston chamber 124 a, and a second fluid pathway 126 b is fluidly coupled between the inlet port 112 and a fluid input 128 b to the second piston chamber 124 b. Similarly, a third fluid pathway 130 a is fluidly coupled between a fluid output 132 a of the first piston chamber and the outlet port 114, and a fourth fluid pathway 130 b is fluidly coupled between a fluid output 132 b of the second piston chamber 124 b and the outlet port 114. As depicted, the first and second fluid pathways 126 a-b may form or be implemented by a y-connector joined at or just before the inlet port 112, and the third and fourth fluid pathways 130 a-b may form or be implemented by a y-connector joined at or just before the outlet port 114.

Each of the fluid inputs 128 a-b and each of the fluid outputs 132 a-b of the first and second piston chambers may include a one-way valve (e.g., a check valve, ball valve, disk valve, or the like), allowing unidirectional flow in the direction of the depicted arrows. That is, the configuration of valves constrains the flow of the fluid such that the fluid can only travel from the inlet port 112 to the fluid inputs 128 a-b of the first and second piston chambers and from the fluid outputs 132 a-b of the first and second piston chambers to the outlet port 114.

In some implementations, the pump cassette 100 may include a bypass valve (or clamp) to divert the fluid that is received from the inlet port 112 directly to the outlet port 114. A lever or button or other control may be located on the housing the perform the bypass. In this regard, the pump cassette 100 may be used to deliver the fluid without the aid of the pistons for a gravity flow delivery.

With respect to the orientation of the pump cassette 100, it is advantageous to have piston chambers 124 a-b vertical and below pistons 122 a-b in order to prevent or limit the impact of air bubbles within the fluid pathway. In some implementations, the top portion of the plunger chamber may allow for capture and/or removal of air bubbles. In this regard, air bubbles may raise to the op of the chamber and capture in the top portion of the plunger chamber. An air valve may be implemented to displace air at the top of the chamber. The pump chambers 124 a-b will first expel any air that is in the pump chamber, thereby preventing air from accumulating in the pump chamber 925. One or more fluid sensors may be disposed within the chamber(s) or along the fluid pathway (e.g., in line 126, 130, 112, or 114). The sensors disposed may include ultrasonic sensors configured as an air-in-line detector.

In some implementations, the cassette drive interface 120 includes a geared coupler configured to mechanically couple to a geared coupler of the pump drive interface. The geared coupler of the cassette drive interface 120 is also mechanically connected, for example, by way of a series of gears 134, to each piston, such that rotation of the gears causes linear reciprocal pumping motion of the pistons. The reciprocating motion mechanism may include a scotch-yoke configuration, a cam-driven (perpendicular motion) configuration, a linear actuator, a rotary actuator, or the like. In some implementations, the cassette drive interface 120 includes an opening in the housing 110 of the cassette 100 to allow insertion of the pump drive interface 116 to directly interface with the system of gears 134 driving the pistons. A rotation of the cassette drive interface 120 is caused by a corresponding rotation of the pump drive interface 116, and the rotation of the cassette drive interface 120 is transferred, by way of the series of gears, into a linear reciprocating motion of the pistons.

According to some implementations, one or more cassette-seated sensors may be disposed within the cassette recess 20 so as to inform central processing unit 12 that the cassette is locked or secured into place within the cassette recess 20 or seat. As described previously, one or more cassette identification features 134 may be coupled to or integrated with the cassette housing at a location configured to interface with identification receiving sensors 118 (cassette-seated sensors) within the cassette recess 20 of the infusion pump system 10. The one or more cassette identification features 134 are configured to, when the pump cassette is seated in the cassette recess, identify to a processor of the infusion pump system 10, via the identification receiving sensors 118, one or more characteristics of the pump cassette (including, e.g., a gear ratio, stroke volume, a range of flow rates, and the like). The cassette identification feature(s) 134 may include, for example, mechanical features such as raised bumps on the housing. Additionally or in the alternative, the cassette identification feature(s) 134 may include an electronic transmitter configured to identify the characteristic(s) of the pump cassette. In this regard, the corresponding identification receiving sensors may include an electronic receiver that receives one or more signals provided by the cassette identification feature(s) 134. In some implementations, the electronic transmitter includes a radio-frequency identification (RFID) tag which communicates with an RFID receiver of the identification receiving feature(s) 118. In some implementations, the electronic transmitter includes a short range wireless transceiver (e.g., Bluetooth) which communicates with a short range wireless transceiver of the identification receiving feature(s) 118.

According to various implementations, the inlet port 112 is configured to fluidly couple to a fluid source 56, which may include a medication container or IV bag, and the outlet port 114 is configured to fluidly couple to an intravenous (IV) infusion set 58. In this regard, that actuation of the cassette drive interface 120 causes the fluid to be removed from the fluid source and delivered through the intravenous infusion set.

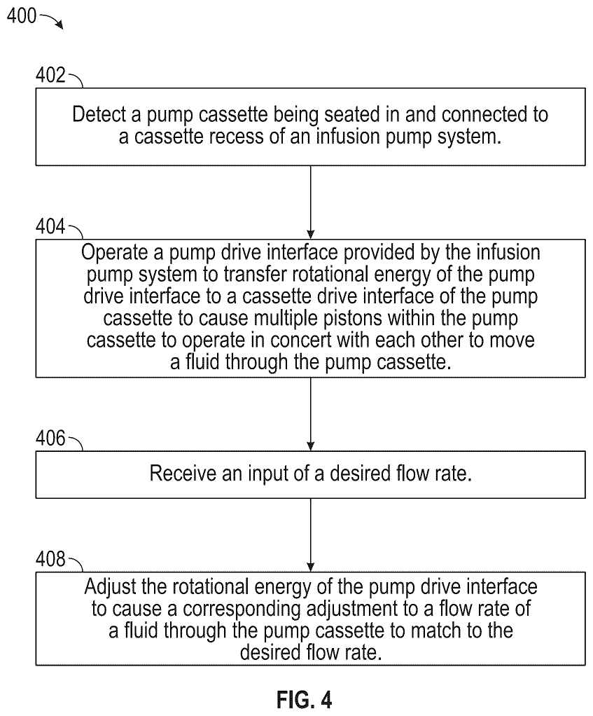

FIG. 4 depicts a first example process 400 for operating a piston-driven cassette based infusion system, according to various aspects of the subject technology. For explanatory purposes, the various blocks of example process 400 are described herein with reference to FIGS. 1-3, as well as the components and processes described herein. In some implementations, one or more of the blocks may be implemented apart from other blocks, and by one or more different processors or devices. Further, for explanatory purposes, the blocks of example process 400 are described as occurring in serial, or linearly. However, multiple blocks of example process 400 may occur in parallel. In addition, the blocks of example process 400 need not be performed in the order shown and one or more of the blocks of example process 400 need not be performed.

According to the depicted example, an infusion pump system 10 (e.g., a processor of the system) detects a pump cassette 100 being seated in an connected to a cassette recess 20 of the infusion pump system 10 (402).

The infusion pump system 10 operates a pump drive interface provided by the infusion pump system to transfer rotational energy of the pump drive interface to a cassette drive interface of the pump cassette to cause multiple pistons within the pump cassette to operate in concert with each other to move a fluid through the pump cassette (404). In this regard, multiple pistons and corresponding piston chambers disposed within the cassette housing and fluidly connected between the inlet port and the outlet port are caused to continuously move a fluid received from the inlet port to the outlet port. In this manner, a first piston of the multiple pistons delivers a first portion of the fluid received into a first piston chamber of the corresponding piston chambers to the outlet port while a second piston of the multiple pistons draws a second portion of the fluid into a second piston chamber of the corresponding piston chambers from the inlet port, and the second piston delivers the second portion of the fluid from the second piston chamber to the outlet port while the first piston draws a third portion of the fluid into the first piston chamber from the inlet port.

According to various implementations, each of the multiple pistons is operatively connected to the cassette drive interface and a controllable fluid pathway is formed between the inlet port and the outlet port and includes the first and second piston chambers. The controllable fluid pathway, including the multiple pistons and piston chambers, are fluidically sealed within the disposable housing 110 of the pump cassette 100, thereby maintaining fluid isolation from the infusion pump system 10 and electronic components therein. In some implementations, the disposable housing 110 may be hermetically sealed.

During operation (or upon startup or initiation of an infusion therapy) the infusion pump system 10 receives an input of a desired flow rate (406). According to various implementations, the input of the desired flow rate may be received, for example, using a touchscreen display screen 14 and/or data input features 16 of the infusion pump system 10. The infusion pump system 10 may then correlate the desired flow rate with a rate of rotation of the pump drive interface. The infusion pump system 10 continues by adjusting the rotational energy of the pump drive interface to cause a corresponding adjustment to a flow rate of the fluid to match the desired flow rate (408). In this regard, the infusion pump system 10 may control a motor associated with the pump drive interface to rotate at the rate of rotation to cause the multiple pistons to pump the fluid through the controllable fluid pathway at the desired flow rate.

Many of the above-described example process 400, and related programming and configuring features, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 5 is a conceptual diagram illustrating an example electronic system 500 for operating a piston-driven cassette based infusion system, according to aspects of the subject technology. Electronic system 500 may be a computing device for execution of software associated with one or more portions or steps of process 500, or components and processes provided by FIGS. 1-6, including but not limited to computing device 8, processor 514, computing hardware within an infusion device 10, or an operably connected remote device (e.g., a mobile device). Electronic system 500 may be representative, in combination with the disclosure regarding FIGS. 1-7. In this regard, electronic system 500 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, an input device interface 514, an output device interface 506, and one or more network interfaces 516. Electronic system 500 further includes a pump motor 718 which is electrically coupled to and controllable by the processing unit(s) 512, and configured to operate the previously described pump drive interface 116. In some implementations, electronic system 500 may include or be integrated with other computing devices or circuitry for operation of the various components and processes previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process, in order to execute the processes of the subject disclosure. The processing unit(s) can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is off. Some implementations of the subject disclosure use a mass-storage device (such as a magnetic or optical disk and its corresponding disk drive) as permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such as a random access memory. System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process in order to execute the processes of some implementations.

Bus 508 also connects to input and output device interfaces 514 and 506. Input device interface 514 enables the user to communicate information and select commands to the electronic system. Input devices used with input device interface 514 include, e.g., alphanumeric keyboards and pointing devices (also called “cursor control devices”). Output device interfaces 506 enables, e.g., the display of images generated by the electronic system 500. Output devices used with output device interface 506 include, e.g., printers and display devices, such as cathode ray tubes (CRT) or liquid crystal displays (LCD). Some implementations include devices such as a touchscreen that functions as both input and output devices.

Also, as shown in FIG. 5, bus 508 also couples electronic system 500 to a network (not shown) through network interfaces 516. Network interfaces 516 may include, e.g., a wireless access point (e.g., Bluetooth or WiFi) or radio circuitry for connecting to a wireless access point. Network interfaces 516 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 500 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. A pump cassette comprising: a cassette housing comprising an inlet port to an outlet port, the cassette housing being configured to be seated in, and connect to, a cassette recess of an infusion pump system; a cassette drive interface configured to interface with a corresponding pump drive interface provided by the infusion pump system when the cassette housing is seated in and connected to the cassette recess; and multiple pistons and corresponding piston chambers disposed within the cassette housing and fluidly connected between the inlet port and the outlet port, each of the multiple pistons being operatively connected to the cassette drive interface and configured to, when the housing is seated in the cassette recess of the infusion pump system and the pump drive interface is actuated by the infusion pump system, operate in concert with each other to continuously move a fluid received from the inlet port to the outlet port, such that a first piston of the multiple pistons delivers a first portion of the fluid received into a first piston chamber of the corresponding piston chambers to the outlet port while a second piston of the multiple pistons draws a second portion of the fluid into a second piston chamber of the corresponding piston chambers from the inlet port, and the second piston delivers the second portion of the fluid from the second piston chamber to the outlet port while the first piston draws a third portion of the fluid into the first piston chamber from the inlet port, wherein a controllable fluid pathway is formed between the inlet port and the outlet port and includes the first and second piston chambers.

Clause 2. The pump cassette of Clause 1, wherein the cassette drive interface comprises a first geared coupler configured to mechanically couple to a second geared coupler of the pump drive interface, wherein a rotation of the cassette drive interface is caused by a corresponding rotation of the pump drive interface, and the rotation of the cassette drive interface is transferred, by way of a series of gears, into a linear reciprocating motion of the multiple pistons.

Clause 3. The pump cassette of Clause 1, wherein the cassette drive interface comprises a motor and is configured to receive power from the pump drive interface to power the motor.

Clause 4. The pump cassette of Clause 1, further comprising: an electronic transmitter configured to identify, to an electronic receiver of the infusion pump system, one or more characteristics of the pump cassette including a gear ratio, stroke volume, and a range of flow rates.

Clause 5. The pump cassette of Clause 1, further comprising: one or more cassette identification features coupled to or integrated with the cassette housing at a location configured to interface with identification receiving sensors within the cassette recess of the infusion pump system, wherein the one or more cassette identification features are configured to, when the pump cassette is seated in the cassette recess, identify to a processor of the infusion pump system, via the identification receiving sensors, one or more characteristics of the pump cassette including a gear ratio, stroke volume, and a range of flow rates.

Clause 6. The pump cassette of Clause 1, further comprising: a first fluid pathway fluidly coupled between the inlet port and a fluid input of the first piston chamber; a second fluid pathway fluidly coupled between the inlet port and a fluid input to the second piston chamber; a third fluid pathway fluidly coupled between a fluid output of the first piston chamber and the outlet port; and a fourth fluid pathway fluidly coupled between a fluid output of the second piston chamber and the outlet port, wherein the fluid inputs and the fluid outputs of the first and second piston chambers each comprise a one way valve such that fluid can only travel from the inlet port to the fluid inputs of the first and second piston chambers and from the fluid outputs of the first and second piston chambers to the outlet port.

Clause 7. The pump cassette of Clause 1, wherein the inlet port is configured to fluidly couple to a fluid source, and the outlet port is configured to fluidly couple to an intravenous infusion set such that actuation of the pump drive interface causes the fluid to be removed from the fluid source and delivered through the intravenous infusion set.

Clause 8. A piston-driven cassette based infusion system comprising: the pump cassette of Clause 1; and the infusion pump system of Claim 1 comprising a pump housing, wherein the pump housing comprises the cassette recess, wherein the infusion pump system is configured to: actuate the pump drive interface to cause the fluid to move through the controllable fluid pathway.

Clause 9. The piston-driven cassette based infusion system of Clause 8, wherein the infusion pump system is configured to: receive an input of a desired flow rate for the fluid traveling through the controllable fluid pathway; correlate the desired flow rate with a rate of rotation of the pump drive interface; and control a motor associated with the pump drive interface to rotate at the rate of rotation to cause the multiple pistons to pump the fluid through the controllable fluid pathway at the desired flow rate.

Clause 10. The piston-driven cassette based infusion system of Clause 9, wherein the cassette recess is configured to receive different sized pump cassettes, and wherein the infusion pump system is further configured to: determine a characteristic of the pump cassette based on a feature of the pump cassette that is readable when the pump cassette is seated in the cassette recess; and adjust, based on the determined characteristic, the rate of rotation of the pump drive interface to cause a corresponding adjustment to a flow rate of the fluid through the controllable fluid pathway of the pump cassette to correspond to the desired flow rate.

Clause 11. The piston-driven cassette based infusion system of Clause 8, wherein the infusion pump system is configured to: detect a resistance of the cassette drive interface; determine an error condition based on the detected resistance; and provide an indication of the error condition.

Clause 12. The piston-driven cassette based infusion system of Clause 11, wherein the infusion pump system is further configured to: activate a pressure sensor responsive to detecting that the resistance satisfies a threshold resistance; measure, when the pressure sensor is activated, a pressure in an infusion line fluidly coupled to the inlet or outlet port; and determine the error condition in part based on a pressure measured by the pressure sensor.

Clause 13. The piston-driven cassette based infusion system of Clause 12, wherein the infusion pump system is further configured to: determine that the pressure satisfies a threshold pressure; and responsive the pressure satisfying the threshold pressure, suspend actuation of the pump drive interface to prevent further pumping of the fluid through the controllable fluid pathway.

Clause 14. The piston-driven cassette based infusion system of Clause 8, wherein the infusion pump system comprises a processor, wherein the cassette recess comprises one or more identification receiving sensors that are configured to, when the pump cassette is seated in the cassette recess, interface with one or more cassette identification features coupled to or integrated with the cassette housing, wherein, when the pump cassette is seated in the cassette recess, the one or more identification receiving sensors cause the processor to identify, based on the one or more cassette identification features, one or more characteristics of the pump cassette including a gear ratio, stroke volume, and a range of flow rates.

Clause 15. A non-transitory machine-readable medium having instructions stored thereon that, when executed by a machine, cause the machine to perform operations comprising: detecting a cassette housing of a pump cassette being seated in, and connected to, a cassette recess of an infusion pump system, the pump cassette comprising an inlet port to an outlet port; operating a corresponding pump drive interface provided by the infusion pump system to transfer a rotational energy to a cassette drive interface of the pump cassette when the cassette housing is seated in and connected to the cassette recess; causing, based on the rotational energy, multiple pistons and corresponding piston chambers disposed within the cassette housing and fluidly connected between the inlet port and the outlet port to operate in concert with each other to continuously move a fluid received from the inlet port to the outlet port, such that a first piston of the multiple pistons delivers a first portion of the fluid received into a first piston chamber of the corresponding piston chambers to the outlet port while a second piston of the multiple pistons draws a second portion of the fluid into a second piston chamber of the corresponding piston chambers from the inlet port, and the second piston delivers the second portion of the fluid from the second piston chamber to the outlet port while the first piston draws a third portion of the fluid into the first piston chamber from the inlet port; receiving an input of a desired flow rate; and adjust the rotational energy of the pump drive interface to cause a corresponding adjustment to a flow rate of the fluid to match the desired flow rate, wherein each of the multiple pistons is operatively connected to the cassette drive interface and a controllable fluid pathway is formed between the inlet port and the outlet port and includes the first and second piston chambers.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. A pump cassette comprising:
a cassette housing comprising an inlet port to an outlet port, the cassette housing being configured to be seated in, and connect to, a cassette recess of an infusion pump system;
a cassette drive interface configured to interface with a corresponding pump drive interface provided by the infusion pump system when the cassette housing is seated in and connected to the cassette recess; and
multiple pistons and corresponding piston chambers disposed within the cassette housing and fluidly connected between the inlet port and the outlet port, each of the multiple pistons being operatively connected to the cassette drive interface and configured to, when the housing is seated in the cassette recess of the infusion pump system and the pump drive interface is actuated by the infusion pump system, operate in concert with each other to continuously move a fluid received from the inlet port to the outlet port, such that a first piston of the multiple pistons delivers a first portion of the fluid received into a first piston chamber of the corresponding piston chambers to the outlet port while a second piston of the multiple pistons draws a second portion of the fluid into a second piston chamber of the corresponding piston chambers from the inlet port, and the second piston delivers the second portion of the fluid from the second piston chamber to the outlet port while the first piston draws a third portion of the fluid into the first piston chamber from the inlet port,
wherein a controllable fluid pathway is formed between the inlet port and the outlet port and includes the first and second piston chambers.

2. The pump cassette of claim 1,
wherein the cassette drive interface comprises a first geared coupler configured to mechanically couple to a second geared coupler of the pump drive interface,
wherein a rotation of the cassette drive interface is caused by a corresponding rotation of the pump drive interface, and the rotation of the cassette drive interface is transferred, by way of a series of gears, into a linear reciprocating motion of the multiple pistons.

3. The pump cassette of claim 1, wherein the cassette drive interface comprises a motor and is configured to receive power from the pump drive interface to power the motor.

4. The pump cassette of claim 1, further comprising:
an electronic transmitter configured to identify, to an electronic receiver of the infusion pump system, one or more characteristics of the pump cassette including a gear ratio, stroke volume, and a range of flow rates.

5. The pump cassette of claim 1, further comprising:
one or more cassette identification features coupled to or integrated with the cassette housing at a location configured to interface with identification receiving sensors within the cassette recess of the infusion pump system,
wherein the one or more cassette identification features are configured to, when the pump cassette is seated in the cassette recess, identify to a processor of the infusion pump system, via the identification receiving sensors, one or more characteristics of the pump cassette including a gear ratio, stroke volume, and a range of flow rates.

6. The pump cassette of claim 1, further comprising:
a first fluid pathway fluidly coupled between the inlet port and a fluid input of the first piston chamber;
a second fluid pathway fluidly coupled between the inlet port and a fluid input to the second piston chamber;
a third fluid pathway fluidly coupled between a fluid output of the first piston chamber and the outlet port; and
a fourth fluid pathway fluidly coupled between a fluid output of the second piston chamber and the outlet port,
wherein the fluid inputs and the fluid outputs of the first and second piston chambers each comprise a one way valve such that fluid can only travel from the inlet port to the fluid inputs of the first and second piston chambers and from the fluid outputs of the first and second piston chambers to the outlet port.

7. The pump cassette of claim 1, wherein the inlet port is configured to fluidly couple to a fluid source, and the outlet port is configured to fluidly couple to an intravenous infusion set such that actuation of the pump drive interface causes the fluid to be removed from the fluid source and delivered through the intravenous infusion set.

8. A piston-driven cassette based infusion system comprising:
The pump cassette of claim 1; and
the infusion pump system of claim 1 comprising a pump housing, wherein the pump housing comprises the cassette recess,
wherein the infusion pump system is configured to:
actuate the pump drive interface to cause the fluid to move through the controllable fluid pathway.

9. The piston-driven cassette based infusion system of claim 8, wherein the infusion pump system is configured to:
receive an input of a desired flow rate for the fluid traveling through the controllable fluid pathway:
correlate the desired flow rate with a rate of rotation of the pump drive interface; and
control a motor associated with the pump drive interface to rotate at the rate of rotation to cause the multiple pistons to pump the fluid through the controllable fluid pathway at the desired flow rate.

10. The piston-driven cassette based infusion system of claim 9, wherein the cassette recess is configured to receive different sized pump cassettes, and wherein the infusion pump system is further configured to:
determine a characteristic of the pump cassette based on a feature of the pump cassette that is readable when the pump cassette is seated in the cassette recess; and
adjust, based on the determined characteristic, the rate of rotation of the pump drive interface to cause a corresponding adjustment to a flow rate of the fluid through the controllable fluid pathway of the pump cassette to correspond to the desired flow rate.

11. The piston-driven cassette based infusion system of claim 8, wherein the infusion pump system is configured to:
detect a resistance of the cassette drive interface;
determine an error condition based on the detected resistance; and
provide an indication of the error condition.

12. The piston-driven cassette based infusion system of claim 11, wherein the infusion pump system is further configured to:
activate a pressure sensor responsive to detecting that the resistance satisfies a threshold resistance;
measure, when the pressure sensor is activated, a pressure in an infusion line fluidly coupled to the inlet or outlet port; and
determine the error condition in part based on a pressure measured by the pressure sensor.

13. The piston-driven cassette based infusion system of claim 12, wherein the infusion pump system is further configured to:
determine that the pressure satisfies a threshold pressure; and
responsive the pressure satisfying the threshold pressure, suspend actuation of the pump drive interface to prevent further pumping of the fluid through the controllable fluid pathway.

14. The piston-driven cassette based infusion system of claim 8,
wherein the infusion pump system comprises a processor,
wherein the cassette recess comprises one or more identification receiving sensors that are configured to, when the pump cassette is seated in the cassette recess, interface with one or more cassette identification features coupled to or integrated with the cassette housing,
wherein, when the pump cassette is seated in the cassette recess, the one or more identification receiving sensors cause the processor to identify, based on the one or more cassette identification features, one or more characteristics of the pump cassette including a gear ratio, stroke volume, and a range of flow rates.

15. A non-transitory machine-readable medium having instructions stored thereon that, when executed by a machine, cause the machine to perform operations comprising:
detecting a cassette housing of a pump cassette being seated in, and connected to, a cassette recess of an infusion pump system, the pump cassette comprising an inlet port to an outlet port;
operating a corresponding pump drive interface provided by the infusion pump system to transfer a rotational energy to a cassette drive interface of the pump cassette when the cassette housing is seated in and connected to the cassette recess;
causing, based on the rotational energy, multiple pistons and corresponding piston chambers disposed within the cassette housing and fluidly connected between the inlet port and the outlet port to operate in concert with each other to continuously move a fluid received from the inlet port to the outlet port, such that a first piston of the multiple pistons delivers a first portion of the fluid received into a first piston chamber of the corresponding piston chambers to the outlet port while a second piston of the multiple pistons draws a second portion of the fluid into a second piston chamber of the corresponding piston chambers from the inlet port, and the second piston delivers the second portion of the fluid from the second piston chamber to the outlet port while the first piston draws a third portion of the fluid into the first piston chamber from the inlet port;
receiving an input of a desired flow rate; and
adjust the rotational energy of the pump drive interface to cause a corresponding adjustment to a flow rate of the fluid to match the desired flow rate,
wherein each of the multiple pistons is operatively connected to the cassette drive interface and a controllable fluid pathway is formed between the inlet port and the outlet port and includes the first and second piston chambers.
